(19) United States
(12) Patent Application Publication
(10) Pub. No.: US 2017/0274027 A1
(43) Pub. Date: Sep. 28, 2017

(54) Cannabis-Hemp Animal Feed

(71) Applicant: David Harold Moore, Henderson, NV (US)

(72) Inventor: David Harold Moore, Henderson, NV (US)

(21) Appl. No.: 15/080,526

(22) Filed: Mar. 24, 2016

(51) Int. Cl. A61K 36/185; A23K 20/158; A23K 40/25; A23K 10/30

(52) U.S. Cl. CPC A61K 36/185; A23K 10/30; A23K 20/158; A23K 40/25

(57) ABSTRACT

The present invention provides a commercial process for preparing the ‘Cannabis-Hemp Animal Feed’ into a feed grade nutritional supplement. The process produces both a fully cooked, and raw feed product combined, that can be inserted into an edible consumption vessel of any type, or processed into any shape, for animal ingestion purposes.

The commercial process entails the combining of various proteins, enzymes, vitamins, minerals, terpenes, flavonoids, decarboxylated cannabinoids (and various raw cannabinoids) that are found in the Cannabis-Hemp Plant; with various fats and thickeners for the purposes of creating a ‘feed’ that can be ingested and metabolized as a feed grade nutritional supplement, and/or as a direct animal feed product.

To accomplish this process, a quantity of the Cannabis-Hemp Plant must be raw and processed, and a quantity of the Cannabis-Hemp Plant must be heat-cooked, with added fats or not. The heat-cooked Cannabis-Hemp plant may then be repeatedly heat-cooked (with fresh fats or not) for maximum efficacy.

### BACKGROUND

My name is David Harold Moore and I am the inventor of the ‘Cannabis Hemp Animal Feed’ process. I have suffered from a High Functioning form of Autism, known as Asperger's Syndrome, since I was born. The autism caused the dysregulation of my endocannabinoid immune system, which caused the auto-immune disorder known as Celiac Disease, which then turned into stomach cancer. And only after going into renal failure, did I begin to look for a solution.

Then I read a recent study from the Biomedical Center for Autism Research and Treatment, which suggests the CB2 receptor as a potential therapeutic target for the cannabis-hemp infused coconut-oil. This study was the first to demonstrate the up-regulation of CB2 receptors in autistic endocannabinoid systems.

The CB2 receptor enhancements demonstrate that the endocannabinoid system is involved in the maintenance of autism. According to the findings, the endocannabinoid system may represent a novel treatment opportunity for cannabis therapy with autistic minds. And that's because the up-regulation of the CB2 receptors enhances the peripheral organs ability to manage the immune system. And endocannabinoid signaling, in general, affects learning, pain, and other important regenerative processes.

You see, the endocannabinoid system is an intricate network of endocannabinoids, their receptors, and the associated enzymes; and all play critical roles in maintaining the body's homeostasis. And our biochemical homeostasis is unique to each one of us. It is therefore important to note, that in most cases, scientists make decisions based on evidence gained from an experiment with extensive controls. But the differences in cannabinoid composition, varies from batch to batch, thus making cannabis-hemp experiments too organic for scientific rigidity.

So I needed to find a healthy and inexpensive way to make a nutritional Cannabis-Hemp food grade dietary supplement. A daily dietary supplement that was all natural and healthy, a capsule with little to no psychoactivity, and a capsule that could be taken in large quantities without harmful side effects. But there were no commercial products that would meet my needs.

Then I began experimenting with different ingredients and different heating methods. This process took about two years before I came up with a novel, non-obvious and useful method to process the Cannabis-Hemp plant into a food grade dietary supplement.

And this novel, non-obvious and useful method is a process based upon the different pressings of the raw and cooked cannabis-hemp plant, combined with various fats, to produce different amalgams that holistically synergize to create the wholesome food product, and nutritional dietary supplement, called ‘Cannabis-Hemp Paste’ (patent Ser. No. 14/969,497).

So after applying for the ‘Cannabis Hemp Paste Patent’, I started researching more about the endocannabinoid system, and found out that all of the animals on this planet also have an endocannabinoid system. Then I had another ‘crystallization moment’, where I realized that the cannabis-hemp plant must be decarboxylated using my previously discovered methods, so our livestock could also realize the efficacy of the cannabis-hemp plant.

I then delved into animal suffering, animal feed, and animal antibiotics; and realized that the ‘Cannabis-Hemp Animal Feed’, if processed correctly, would reduce animal suffering, eliminate the need for animal antibiotics, and reduce the need for arable farmland, to feed our livestock with.

### SUMMARY

The ‘Cannabis-Hemp Animal Feed’ process is a novel, non-obvious and useful method to transform the Cannabis-Hemp Plant into an effective feed grade nutritional supplement.
A primary object of the present invention is to provide an inexpensive method to commercially process the Cannabis-Hemp plant into a feed grade nutritional supplement, so that the process overcomes the shortcomings of the other methods.
Another object is to provide a whole plant supplement to reduce the amount of wasted Cannabis-Hemp material that is being thrown away after current harvesting methods.
An additional object is to provide a feed product that can be stored for extended periods of time, to process that feed product into feed pellet shapes that animals like, and to package the feed product into large feed bags so as to replace GMO Corn as the staple animal feed.
Another object is to provide an affordable Cannabis-Hemp feed grade nutritional supplement that can be ingested regularly, so as to reduce the need for animal antibiotics.

Further objects of the invention will appear as the description proceeds . . .

Briefly described, in its preferred form, the present invention provides a method for preparing a commercially inexpensive way to produce a high quality Cannabis-Hemp Animal Feed. The present process preferably comprises the following steps:
a) providing a quantity of the raw cannabis hemp plant;
b) providing a system to convey the raw feed product through the process;
c) heat-cooking the raw cannabis-hemp flowers to decarboxylate a quantity of cannabinoids
d) adding and applying various fresh fats and oils to the previously cooked cannabis-hemp flowers;
e) cooking the previously cooked hemp flowers for the second time, for further decarboxylation
f) adding the raw cannabis-hemp leaves, and raw cannabis-hemp seeds, to twice cooked flowers.
g) blending fillers, and other animal feeds, to the twice cooked, processed, and raw feed product.
h) processing and compressing the combined feed product into various shapes for animal ingestion.

A raw feed product at ambient temperature is the preferred initial feed product for the present process. That is, the most desirable final Cannabis Hemp Animal Feed product begins with a raw product in the beginning of the process. While an initially raw/fresh initial product is preferred, the present process can accommodate an initial feed product that has been frozen and dried, that is above or below ambient temperature, and that has already been dehumidified.

The heat-cooking temperature and the time period the product stays within the cooking chamber can be adjusted for the various decarboxylation processes, that the present invention is designed to handle. Each component step of the present invention can similarly be adjusted. Some of the adjustments can be incorporated by simply manipulating heat times, and heat temperaturs.

The preferable heat-cooking steps may comprise only one dry heat cooking step, multiple dry heat-cooking steps, and/or a dry heat cooking step with applied fat heat cooking steps. Yet if the product is only cooked with heat, and no applied fats, the final product typically lacks the amount of decarboxylated cannabinoids of the heat-cooked product, with added fats. If the product is only heated without added fats, or without the multiple heat cooking steps, the process will produce an inferior product.

In the conventional processing methods, the process incorporates only using the raw leaves from the Cannabis-Hemp Plant, and none of the heat cooked Cannabis-Hemp Flowers, and none of the processed or raw Cannabis-Hemp Seeds. One of the problems with this conventional process is that by using only raw cannabinoids, the animals can't realize the full efficacy of the decarboxylated cannabinoids.

In the present process, the fats are applied after convection cooking for the first time, and a raw portion of the final feed product is never heated so as to preserve the naturally occurring enzymes, minerals, vitamins, terpenes, terpenoids, and flavonoids. Thus, the product retains its full compliment of healthy ingredients because differently processed plant materials are added throughout the various blending steps; to create both the enhanced, and superior product, known as ‘Cannabis-Hemp Animal Feed’.

If the feed product is too hot at any point in the process, then many of the cannabinoids will be lost. If the feed product is too cool during the heat-cooking process, then the cannabinoids won't decarboxylate and the product won't be nearly as effective.

Unlike the conventional processes, the present invention combines both raw and cooked aspects of the cannabis-hemp plant, to the final Cannabis-Hemp Animal Feed. Thus upon ingesting, the biochemical affects can help upregulate the endocannabinoid immune system, which then helps provide a biochemical homeostasis, which can then help our livestock self-heal through a daily healthful feeding regimen.

The present invention enables a single location to process, cure, store, cook and package the ‘Cannabis-Hemp Animal Feed’ in large quantities. Therefore, each retail outlet can eliminate any reliance upon, and the capital expense for, third party quality certifications. They need simply to refer them to the publicly published materials, and the transparent process, being shared by the current patent application owner.

Finally, the novel and useful ‘Cannabis-Hemp Animal Feed’ process, utilizes several steps in the production of the final ‘feed grade nutritional supplement’. Accordingly, an object of the invention is to provide a commercial process for preparing of an affordable Cannabis Hemp Animal Feed that can be ingested daily, and with no harmful side effects.

### DETAILED DESCRIPTION

The present invention provides a commercial process for preparing Cannabis-Hemp Paste. The process produces both a fully cooked and raw feed product that can be compressed into a consumption shape of any type.

To process the Cannabis-Hemp Animal Feed, the steps include:
providing a quantity of raw cannabis-hemp (flowers, stems, seeds, and leaves); providing a means to convey the raw cannabis-hemp product through the various steps of the present invention; grinding the cannabis-hemp plant; grinding other feed sources and fillers to be added later; processing the seeds to extract oil and flour; heat cooking the raw flowers; adding hemp seed oil to the once heat-cooked flowers; re-heat cooking the previously cooked flowers with the added hemp seed oil; blending the correct ratio of raw leaves, seed flour, seed oil, cooked flowers and stems, with the other ground animal feed sources; compressing the final product into various shapes and sizes for animal ingestion purposes; drying and cooling the final product; crumbling the final product (based on animal preference); and packaging the final product for proper storage.

### CANNABIS-HEMP ANIMAL FEED ABSTRACT RECIPE

Assemble Initial Ingredients:

Provide the Raw Cannabis-Hemp Plant (flowers, seeds, stems and leaves)

Add Various Fats to the Cannabis-Hemp Flower (hemp seed oil)

Add Other Sources of Animal Feed (seed meal, flour, grains, vegetables, etc.)

Phase One (Initial Processing)

Disassemble Cannabis-Hemp Plant Stalks

Remove and Air Dry Leaves

Remove and Process Seeds into Oil and Seed Meal (Flour)

Removes Flowers and Stems from Stalks

Grind Flowers, Stems, and Leaves through the Hammer Mill

Phase Two (Flower Decarboxylation).

Decarboxylize Flowers in Convection Oven

Add Fats to Decarboxylized Flowers

Decarboxylize Flowers in Convection Oven—for second time

Phase Three (Final Blending)

Blend the Grounded: Raw Leaves, Heat Cooked flowers, and Seed Meal together

Blend Other Sources of Animal Feed with the previously blended Feed Product

Process the Final Blended Animal Feed through the Pellet Mill

Phase Four (Drying and Cooling)

Now take the completed Animal Feed Pellets and dry in the Dryer Cooling Machine.

and now crack the pellets, but only if the specific animals prefer crumbled animal feed pellets

1. A process for preparing an effective ‘cannabis-hemp animal feed’ comprising the ordered steps of:
a) providing a quantity of the raw cannabis-hemp plant (‘the feed product’)
b) providing a system to convey the feed product through the various steps of the process
c) preparing the raw cannabis-hemp feed product, by grinding it for feed processing
d) grinding other nutritional feed sources, and fillers, for later additions to the end feed product
e) preparing the raw cannabis-hemp feed product by processing the seeds to extract the oil
f) preparing the raw cannabis-hemp by processing the processed seed hulls into a hemp flour
g) applying the processed hemp seed oil onto the hemp flowers before heat cooking
h) preparing the feed product by heat cooking the ground hemp flowers (with the added seed oil)
i) choosing proper ratios of the heat cooked, raw, and processed cannabis-hemp feed product
j) blending the proper ratios of the heat cooked, raw, and processed cannabis-hemp feed product
k) choosing the proper ratio blends, of the other animal feed products, to be added
l) blending other feed sources with the heat cooked, raw, and processed cannabis-hemp feed product
m) choosing the most effective feed size and shape (depending on animal preference)
n) choosing the appropriate dye, or shape, for the feed processing equipment
o) processing the feed product through the feed processing equipment
p) cooling and drying the feed product for proper storage
q) crumbling or cracking the feed pellets, depending on animal preference

2. The process of preparing ‘cannabis-hemp animal feed’ according to claim 1, wherein the feed product is fully prepared upon exiting the steps of heat cooking the flowers (i) and wherein the feed product is fully prepared after blending the heat cooked, raw, and processed cannabis-hemp feed product (j) together; with or without the additional animal feed sources.

3. The process of preparing ‘cannabis-hemp animal feed’ according to claim 1, wherein the feed product is fully prepared, without processing the cannabis-hemp seeds (e, f, g) but adding the whole raw seeds, and wherein the feed product is fully prepared upon exiting the steps of heat cooking the cannabis-hemp flowers (i) and after blending the heat cooked, raw, and processed cannabis-hemp feed product (j) together; with or without the other additional animal feed sources.

4. A twice cooked process for preparing ‘cannabis-hemp animal feed’ according to claim 1, comprising the steps of:
a. providing a quantity of the raw cannabis hemp plant;
b. providing a system to convey the raw feed product through the process;
c. heat-cooking the raw cannabis-hemp flowers to decarboxylate a quantity of cannabinoids
d. adding and applying various fresh fats and oils to the previously heat cooked flowers;
e. cooking the previously cooked hemp flowers for the second time, for further decarboxylation
f. adding the raw cannabis-hemp leaves, and raw cannabis-hemp seeds, to twice cooked flowers.
g. blending fillers, and other animal feeds, to the twice cooked, processed, and raw feed product.

5. The process of preparing ‘cannabis-hemp animal feed’ according to claim 1 through 4, comprising the further step of combining the feed product with various spices, seeds, nuts, roots, flavorings, condiments, fruits, herbs, flowers, leaves, vegetables, extracts, and sweeteners before and after steps (c to q).

6. The process of preparing ‘cannabis-hemp animal feed’ according to claims 1 through 5, comprising the further step of freezing and or refrigerating the feed product before and after each step (a to q).

7. The process of preparing ‘cannabis-hemp animal feed’ according to claims 1 through 6, wherein the step of blending the feed product (j, l, o) comprises the sub-steps of:
(i) applying a thickener to the feed product
(ii) removing liquids from the feed product to thicken.
